(19) United States
(12) Patent Application Publication
(10) Pub. No.: US 2024/0050466 A1
(43) Pub. Date: Feb. 15, 2024

(54) CELLULAR HEALTH NUTRITIONAL PRODUCT

(71) Applicant: GLAXOSMITHKLINE CONSUMER HEALTHCARE HOLDINGS (US) LLC, Wilmington, DE (US)

(72) Inventors: Caroline Felicity BULL, Adelaide, , South Australia (AU); Michael FENECH, North Brighton, South Australia (AU); Scott William POXON, Richmond, VA (US); Bernardus Jan-Willem VAN KLINKEN, Warren, NJ (US)

(21) Appl. No.: 18/258,101

(22) PCT Filed: Dec. 20, 2021
(86) PCT No.: PCT/US2021/064340

§ 371 Date: Jun. 16, 2023

Related U.S. Application Data
(60) Provisional application No. 63/128,904, filed on Dec. 22, 2020.

(51) Int. Cl. A61K 33/04; A61K 31/375; A61K 31/355; A61K 31/4172; A61K 31/12; A61K 31/7048; A61K 31/01; A61K 31/047

(52) U.S. Cl. CPC A61K 33/04; A61K 31/375; A61K 31/355; A61K 31/4172; A61K 31/12; A61K 31/7048; A61K 31/01; A61K 31/047

(57) ABSTRACT

A nutritional supplement composition formulated having antioxidant and anti-inflammatory nutrients reduces cellular biomarkers of DNA damage and improves cellular regenerative capacity.

### BACKGROUND

### 1. Field of the Disclosure

The present disclosure relates to a cellular protection formulation. More particularly, the present disclosure relates to such a cellular protection formulation having antioxidant and anti-inflammatory nutrients for reducing cellular biomarkers of DNA damage and improving cellular regenerative capacity.

### 2. Brief Description of Related Art

As a person ages, the person's body shows signs of aging. Signs of aging include genomic instability, telomere attrition, epigenetic alterations, loss of proteostasis, deregulated nutrient sensing, mitochondrial dysfunction, cellular senescence, stem cell exhaustion, and altered intercellular communication.

People are living longer. The burden of age-related diseases is increasing. Also, the cost of medical intervention and the associated social and personal burden is becoming unsustainable. Therefore, there is an increasing need to address the fundamental pathologies that cause age-related diseases.

Increased DNA damage at the chromosomal, telomere, mitochondrial DNA, DNA base and DNA methylation levels are all associated with increased risk of developmental and degenerative disease and accelerated aging. Further, preventing the excessive accumulation of DNA damage can by facilitated by optimal diet and lifestyle throughout the various stages of an individual and is important to health and well-being.

Maintenance of genome integrity is dependent on good nutrition to provide the substrates and cofactors required for a cell to synthesize nucleotides, accurately replicate and repair DNA, as well as prevent endogenous and exogenous genotoxicants from harming the genome. Over the past two decades, it has become evident that overt deficiency, as well as sub-clinical deficiencies in micronutrients involved in these processes, can cause as much genome damage as that induced by environmental and lifestyle genotoxins. Also, these deficiencies increase susceptibility to these hazards.

Prospective studies have consistently shown that a higher rate of chromosomal damage, such as micronuclei and chromosome breaks or rearrangements, as well as telomere shortening, predict an increased risk for age-related diseases such as cancer and cardiovascular disease.

It has been demonstrated in several intervention studies that chromosomal DNA damage and telomere shortening can be reduced by appropriate dietary change and/or micronutrient supplementation. Healthy aging at the most fundamental level can thus be promoted by optimizing micronutrient intake.

### SUMMARY

The present disclosure provides a nutritional supplement composition formulated to protect cellular health at the DNA level.

The present disclosure, in addition, provides such a nutritional supplement composition formulated to protect cellular health that preferably reduces the rate of occurrence of cellular biomarkers of DNA damage and improves cellular regenerative capacity.

The present disclosure also provides a nutritional supplement composition that has micronutrients essential for lessening the rate of cellular damage to DNA, increasing antioxidant protection at the cellular level, and mitigating damage caused by intracellular inflammatory responses, and preferably a reduction in reactive nitrogen species (RNS) and/or reactive oxygen species (ROS).

The present disclosure still further provides a nutritional supplement composition that has a combination of micronutrients selected to promote genome integrity and, preferably, cellular health and regenerative capacity.

The present disclosure yet further provides such a nutritional supplement composition that includes the following: Vitamin E, Selenium, and Vitamin C, alone, or in combination with one or more of: carnosine, curcumin, hesperidin, lycopene and lutein.

The present disclosure provides a method for lessening the rate of cellular damage to DNA for the subject, comprising administering such a such a nutritional supplement composition that includes the following: Vitamin E, Selenium, and Vitamin C, alone, or in combination with one or more of: carnosine, curcumin, hesperidin, lycopene and lutein.

The present disclosure provides a method for reducing reactive nitrogen species (RNS) and/or reactive oxygen species (ROS) present in the subject, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for ameliorating a cellular biomarker of DNA damage in the subject, comprising administering such a nutritional supplement composition that includes the following: Vitamin E, Selenium, and Vitamin C, alone, or in combination with one or more of: carnosine, curcumin, hesperidin, lycopene and lutein. The present disclosure further provides wherein the cellular biomarker is at least one selected from the group consisting of: Micronucleus (MN) frequency in lymphocytes and buccal cells, TL in lymphocytes and buccal cells, nucleoplasmic bridges, and nuclear buds.

The present disclosure provides a method for increasing TL in lymphocytes and buccal cells, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for reducing Micronucleus (MN) frequency in lymphocytes and buccal cells comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for reducing abnormal cellular methylation of DNA comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for increasing lipid homeostasis, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for decreasing abnormal cell division, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for decreasing protein oxidation, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for decreasing DNA oxidation and breakage, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for decreasing lipid peroxidation, comprising administering such a nutritional supplement composition that includes the following: Vitamin E, Selenium, and Vitamin C, alone, or in combination with one or more of: carnosine, curcumin, hesperidin, lycopene and lutein.

The present disclosure provides a method for decreasing cellular inflammation, comprising administering such a nutritional supplement composition that includes the following: Vitamin E, Selenium, and Vitamin C, alone, or in combination with one or more of: carnosine, curcumin, hesperidin, lycopene and lutein.

In such a nutritional supplement composition, Vitamin E can be present in an amount from 0.3 mg to 1000 mg, preferably in an amount from 23 mg to 502 mg, and more preferably in an amount from 60 mg to 200 mg.

In such a nutritional supplement composition, Selenium can be present in an amount from 1.1 mcg to 400 mcg, preferably in an amount from 11 mcg to 257 mcg, and more preferably in an amount from 45 mcg to 150 mcg.

In such a nutritional supplement composition, Vitamin C can be present in an amount from 1.5 mg to 2000 mg, preferably in an amount from 15 mg to 1000 mg, and more preferably in an amount from 75 mg to 250 mg

In such a nutritional supplement composition, Carnosine can be present in an amount from 20 mg to 10,000 mg, preferably in an amount from 100 mg to 5000 mg, and more preferably in an amount from 300 mg to 1000 mg

In such a nutritional supplement composition, Curcumin can be present in an amount from 10 mg to 5000 mg, preferably in an amount from 50 mg to 1000 mg, and more preferably in an amount from 150 mg to 500 mg.

In such a nutritional supplement composition, Hesperidin can be present in an amount from 5 mg to 2500 mg, preferably in an amount from 15 mg to 1500 mg, and more preferably in an amount from 75 mg to 250 mg.

In such a nutritional supplement composition, Lycopene can be present in an amount from 0.2 mg to 120 mg, preferably in an amount from 1.2 mg to 52 mg, and more preferably in an amount from 4 mg to 12 mg.

In such a nutritional supplement composition, Lutein can be present in an amount from 0.2 mg to 120 mg, preferably in an amount from 1.2 mg to 52 mg, and more preferably in an amount from 4 mg to 12 mg.

Among the countless known vitamins, minerals, peptides and phytonutrients, the combination of nutrients of the present disclosure, namely Vitamin E, Selenium, and Vitamin C, alone, or in combination with one or more of carnosine, curcumin, hesperidin, lycopene, and lutein, has been found surprisingly effective to promote genome integrity and, preferably, cellular health and regenerative capacity based on the level of cellular damage in humans DNA. The cellular health and regenerative capacity are measured with certain biomarkers that increase with age and oxidative stress and that are modifiable by dietary intervention.

These biomarkers include: (1) chromosome aberrations (in lymphocytes), (2) micronuclei (in lymphocytes and buccal cells), (3) DNA strand breaks measured by “comet” assay (in lymphocytes/leukocytes), (4) DNA oxidation measured as oxidized DNA bases (in lymphocytes/leukocytes) and (5) telomere length or telomerase activity (in lymphocytes/leukocytes). These biomarkers have been found to be the best validated biomarkers of genome integrity.

Without wishing to be bound by a single theory, the nutritional supplement composition of the present disclosure protects genome integrity by the mechanism of micronutrients that essential promote antioxidant and anti-inflammatory responses at the cellular level to enable optimal maintenance of chromosomal and telomere DNA integrity.

### BRIEF DESCRIPTION OF THE DRAWINGS

FIG. 1 is a summary of ingredients and mechanisms of action according to the present disclosure.

FIG. 2 shows examples of cellular damage to DNA.

FIG. 3 is a summary of the effect of supplementation with a formulation according to the present disclosure.

FIG. 4 is another summary of the effect of supplementation with a formulation according to the present disclosure.

FIG. 5 is yet another summary of the effect of supplementation with a formulation according to the present disclosure.

### DETAILED DESCRIPTION OF THE DISCLOSURE

It has been found by the present disclosure that it is possible lessen the rate of cellular damage to DNA, increase antioxidant protection at the cellular level, and mitigate cellular damage to DNA caused by intracellular inflammatory responses by increasing the supply of a certain combination of micronutrients.

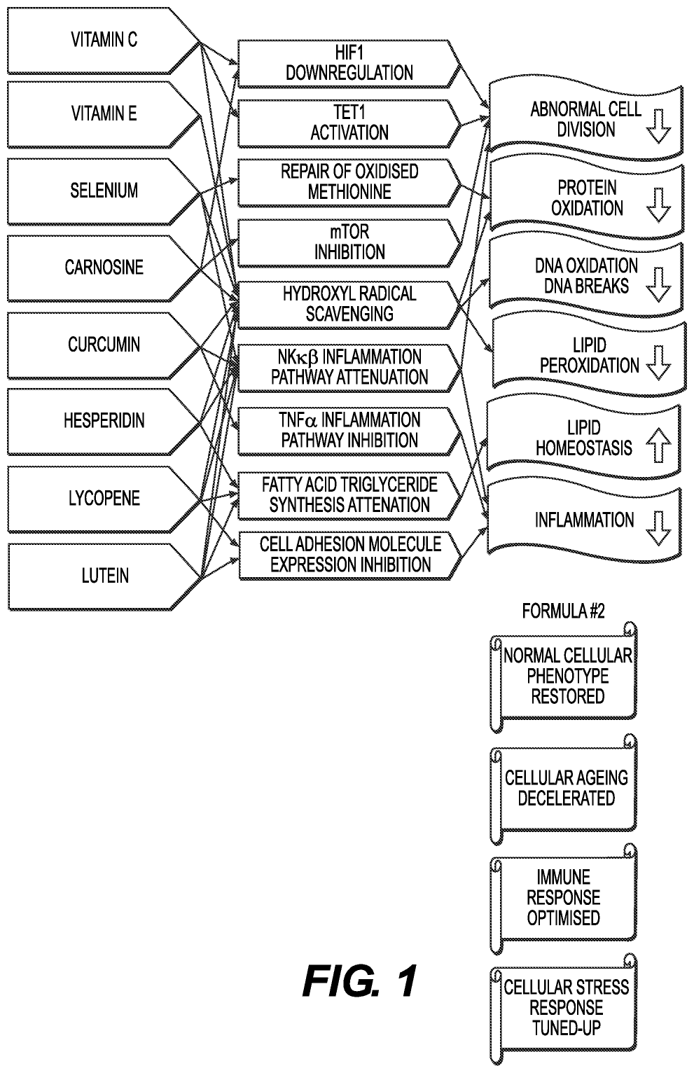

Referring to FIG. 1, the combination of nutrients according to the present disclosure includes: Vitamin E, Selenium, Vitamin C, carnosine, curcumin, hesperidin, lycopene and lutein.

The combination has strong antioxidant and anti-inflammatory effects that can help reduce DNA damage. Moreover, the ingredients in the combination complement each other across nine different mechanisms relating to prevention of oxidative damage and inflammation.

The combination supports HIF1 down-regulation, TET1 activation, oxidized methionine repair, mTOR inhibition, hydroxyl radical scavenging, NFκβ inflammation pathway inhibition, TNα inflammation pathway inhibition, fatty acid triglyceride attenuation, and cell adhesion molecule expression inhibition.

The combination increases lipid homeostasis. The combination also decreases abnormal cell division, protein oxidation, DNA oxidation and break, lipid peroxidation, and cellular inflammation.

Thus, the combination restores normal cellular phenotype, decelerates cellular aging, optimizes immune response and tunes up cellular stress responses.

All the micronutrients in the combination have hydroxyl radical scavenging which leads to prevention of DNA oxidation, DNA breaks, lipid peroxidation and protein oxidation.

Vitamin E, curcumin, lycopene, lutein, hesperidin are lipid soluble and therefore are more efficacious in prevention of oxidative damage in membranes or lipid droplets or chylomicrons compared to Vitamin C, antioxidant selenoenzymes, and carnosine that are hydrophilic micronutrients. These hydrophilic micronutrients exert their antioxidant effects in the aqueous milieu of the cytoplasm, nucleoplasm, tissue interstitial fluids and the circulatory system. Furthermore, selenoenzymes, glutathione peroxidases and thioredoxin reductases, play a key role in maintaining the glutathione antioxidant system and maintaining disulphide bonds in proteins in a reduced state, respectively, while MsrB1 specifically repairs methionine sulphoxide in proteins.

The combination enhances the anti-inflammatory effect of curcumin, hesperidin, lycopene and lutein operating via attenuation of the NFκβ and TNFα pathways as well as by inhibition of cell adhesion molecule expression.

In addition, the combination has cancer growth inhibition properties via down-regulation of HIF1 by both Vitamin C and carnosine that also inhibits mTOR, another growth regulator of tumor cells, and the facilitation of TET-1 activation by Vitamin C that could reverse silencing of tumor suppressor genes in cancer-initiated cells. These diverse combined antioxidant and anti-inflammatory effects, together the improved lipid homeostasis, enable the combination to enhance cellular stress-response, maintenance of normal cellular phenotype and deceleration of cellular aging.

A nutritional supplement composition according to the present disclosure includes one or more vitamins, minerals, peptides, and/or phytonutrients.

Vitamin E can be present in an amount from 0.3 mg to 1000 mg, preferably in an amount from 23 mg to 502 mg, and more preferably in an amount from 60 mg to 200 mg.

Selenium can be present in an amount from 1.1 mcg to 400 mcg, preferably in an amount from 11 mcg to 257 mcg, and more preferably in an amount from 45 mcg to 150 mcg.

Vitamin C can be present in an amount from 1.5 mg to 2000 mg, preferably in an amount from 15 mg to 1000 mg, and more preferably in an amount from 75 mg to 250 mg

Carnosine can be present in an amount from 20 mg to 10,000 mg, preferably in an amount from 100 mg to 5000 mg, and more preferably in an amount from 300 mg to 1000 mg

Curcumin can be present in an amount from 10 mg to 5000 mg, preferably in an amount from 50 mg to 1000 mg, and more preferably in an amount from 150 mg to 500 mg.

Hesperidin can be present in an amount from 5 mg to 2500 mg, preferably in an amount from 15 mg to 1500 mg, and more preferably in an amount from 75 mg to 250 mg.

Lycopene can be present in an amount from 0.2 mg to 120 mg, preferably in an amount from 1.2 mg to 52 mg, and more preferably in an amount from 4 mg to 12 mg.

Lutein can be present in an amount from 0.2 mg to 120 mg, preferably in an amount from 1.2 mg to 52 mg, and more preferably in an amount from 4 mg to 12 mg.

In an exemplary composition or embodiment, Vitamin E is in an amount from 0.3 mg to 1000 mg; Selenium is in an amount from 1.1 mcg to 400 mcg; Vitamin C is in an amount from 1.5 mg to 2000 mg; carnosine is in an amount from 20 mg to 10,000 mg; curcumin is in an amount from 10 mg to 5000 mg; hesperidin is in an amount from 5 mg to 2500 mg; lycopene is in an amount from 0.2 mg to 120 mg; and lutein is in an amount from 0.2 mg to 120 mg.

In another exemplary composition, Vitamin E is in an amount from 0.3 mg to 1000 mg; Selenium is in an amount from 1.1 mcg to 400 mcg; and Vitamin C is in an amount from 1.5 mg to 2000 mg. Optionally, one or more of the following are present: carnosine in an amount from 20 mg to 10,000 mg; curcumin in an amount from 10 mg to 5000 mg; hesperidin in an amount from 5 mg to 2500 mg; lycopene in an amount from 0.2 mg to 120 mg; and lutein is in an amount from 0.2 mg to 120 mg.

In still another exemplary composition, the following are present: Vitamin E in an amount of about 60 mg or about 200 mg; Selenium in an amount of about 45 mcg or about 150 mcg; Vitamin C in an amount of about 75 mg or about 250 mg; carnosine in an amount of about 300 mg or about 1000 mg; curcumin in an amount of about 150 mg or about 500 mg; hesperidin in an amount of about 75 mg or about 250 mg; lycopene in an amount of about 4 mg or about 12 mg; and lutein in an amount of about 4 mg or about 12 mg.

In yet another exemplary composition, the following are present: Vitamin E in an amount of about 60 mg or about 200 mg; Selenium in an amount of about 45 mcg or about 150 mcg; and Vitamin C in an amount of about 75 mg or about 250 mg. Optionally, one or more of the following are present: carnosine in an amount of about 300 mg or about 1000 mg; curcumin in an amount of about 150 mg or about 500 mg; hesperidin in an amount of about 75 mg or about 250 mg; lycopene in an amount of about 4 mg or about 12 mg; and lutein in an amount of about 4 mg or about 12 mg. As used in the embodiments of this paragraph, the term “about” means plus or minus 15%, preferably plus or minus 10%, more preferably plus or minus 7%, and most preferably plus or minus 5%.

These combinations of micronutrients have been surprisingly found to be effective at reducing DNA damage biomarkers and/or improving cellular regenerative capacity in individuals with clinical deficiency in the micronutrients. For example, this combination of micronutrients plays an important role in maintenance of DNA methylation patterns and restoration. Moreover, it has been unexpectedly found that the combination can ameliorate abnormal nucleotides and DNA replication stress resulting in efficient nucleotide synthesis. Further, the combination can ameliorate (1) stalling of DNA replication and increases in DNA breaks resulting in high fidelity DNA replication, (2) hypomethylated dysfunctional centromeres and abnormal gene expression resulting in proper maintenance of DNA methylation, (3) abnormally long or short dysfunctional telomeres resulting in normal telomere length maintenance, (4) aneuploidy that is an abnormal chromosome number resulting in accurate chromosomes segregation, (5) misrepair and/or lack of repair of DNA breaks or DNA adducts resulting in efficient and/or accurate DNA repair, and (6) silencing of DNA repair and cell cycle genes by DNA hypermethylation resulting in reversal of DNA hypermethylation.

Referring to the combination, Vitamin C prevents oxidative damage to DNA and helps regenerate Vitamin E after it is oxidized.

The term “Vitamin C” is intended to include all forms of Vitamin C, such as ascorbic acid dehydroascorbic acid, calcium ascorbate, sodium ascorbate, and other salts of ascorbic acid.

Vitamin C is an essential dietary nutrient for the biosynthesis of collagen and a cofactor in the biosynthesis of catecholamines, L-carnitine, cholesterol, amino acids, and some peptide hormones.

The ability of ascorbate to donate electrons enables it to act as a free-radical scavenger and to reduce higher oxidation states of iron to Fe2+. These reactions are the basis of its biological activity along with the relative stability of the resulting resonance-stabilized monodehydroascorbate radical. The iron reducing activity of ascorbate maintains the reactive center Fe2+ of 2-oxoglutarate-dependent dioxygenases (2-ODDs) thus preventing inactivation. Examples OF 2-ODDs are prolyl hydroxylases, which play a role in the biosynthesis of collagen and in down-regulation of hypoxia-inducible factor (HIF)-1, a transcription factor that regulates many genes responsible for tumor growth, energy metabolism, and neutrophil function and apoptosis. Vitamin C-dependent inhibition of the HIF pathway may provide alternative or additional approaches for controlling tumor progression, infections, and inflammation. As an antioxidant, Vitamin C provides protection against oxidative stress-induced cellular damage by scavenging of reactive oxygen species, by Vitamin E-dependent neutralization of lipid hydroperoxyl radicals, and by protecting proteins from alkylation by electrophilic lipid peroxidation products.

Vitamin C is a provider of reduced iron and is also an essential factor for the function of epigenetic regulators that initiate the demethylation of DNA and histones which is essential for erasure of epigenetic memory during early development via ten-eleven translocation (TET) enzymes that catalyze oxidation of 5-methylcytosine which is then replaced by unmethylated cytosine. The Jumonji C (JmjC)-domain-containing histone demethylases also require ascorbate as a cofactor for histone demethylation. Thus, by primarily participating in the demethylation of both DNA and histones, ascorbate appears to play an important role in epigenome integrity maintenance.

Lack of Vitamin C causes scurvy, a pathological condition leading to blood vessel fragility and connective tissue damage due to failure in producing collagen. Such scurvy eventually leads to death as result of a general collapse.

Vitamin C facilitates cancer and cardiovascular diseases prevention.

Higher Vitamin C intake is associated with reduced risk for cardiovascular diseases. Vitamin C has stronger effects in those at higher cardiovascular disease risk that in healthy individuals.

However, there is no evidence that Vitamin C supplementation alone impacts the risks for all-cause mortality, impaired cognitive performance, reduced quality of life, the development of eye diseases, infections, cardiovascular disease, and cancers in populations already adequately supplied through dietary sources. Furthermore, dietary intake might not be the sole determinant of systemic concentrations, since variations in genes participating in redox homeostasis and Vitamin C transport have been associated with lowered plasma concentrations. The majority of placebo-controlled intervention studies investigating effects of Vitamin C supplementation on its own, performed in well-nourished participants, showed a null-effect on DNA damage. Protective effects of Vitamin C were only observed in those with low dietary intake of Vitamin C, a genetic defect in uptake of Vitamin C into cells or when it was taken in combination with other micronutrients (e.g. Vitamin E, Vitamin A) and intervention periods of 12 weeks or greater. Thus, it is believed that any genome protective effects of Vitamin C occur via indirect mechanisms (e.g., inhibition of lipid peroxidation) such as in the combination of the present disclosure, and be more evident in those who are Vitamin C deficient due to low dietary intake or genetic defects in Vitamin C uptake into cells.

In the examples herein, Vitamin C is in the form of ascorbic acid (pure), found naturally in plants and mammals.

A dosage of 250 mg/day is enough to saturate Vitamin C levels in plasma and cells even in those with low intake from foods.

Vitamin E prevents formation of lipid hydroperoxides and DNA damage caused by lipid hydroperoxides.

The term “Vitamin E” is intended to include all functionally equivalent forms of tocopherol including d-alpha-tocopherol, d-beta tocopherol, d-gamma tocopherol, and d-delta tocopherol. The term “Vitamin E” is also intended to include all functionally equivalent forms of tocotrienols including alpha tocotrienols, beta tocotrienols, gamma tocotrienols, and delta tocotrienols.

Vitamin E functions as an essential lipid soluble antioxidant, scavenging hydroperoxyl radicals in the lipid milieu of cell membranes and body fluids. As a result, it prevents the formation of lipid peroxides and hydroperoxides that cause membrane malfunction and fragility. In addition, lipid peroxides and hydroperoxides react and adduct with DNA, RNA and proteins leading to cellular mutation and accelerated aging. The capacity of Vitamin E to prevent lipid peroxidation is dependent on the free-radical scavenging of Vitamin C and glutathione and antioxidant enzymes such as the Selenium-enzyme glutathione peroxidase (GPX4).

Vitamin E, in phosphorylated form, has been shown to affect cell signal transduction and gene expression, both in vitro and in vivo. Phosphorylation of Vitamin E, which takes place in vivo, results in a molecule provided with functions that are in part stronger and in part different from those of the non-phosphorylated compound.

Through its preventive effects against lipid peroxidation and lipid peroxide damage of cellular macromolecules Vitamin E plays a significant role in protecting against cancer, cardiovascular disease, neurodegenerative diseases, diabetes, chronic liver failure, immune dysfunction, inflammation and cataracts. Other in vivo documented functions of Vitamin E include preventing Vitamin E deficiency ataxia and slowing down the progression of non-alcoholic steato-hepatitis (NASH).

In the examples, Vitamin E is in the form of tocopherols and tocotrienols.

Such forms have positive protective effects with regards to reduction in lipid peroxidation in vivo, resistance to lipid peroxidation ex vivo, improvements in immune function and in DNA damage prevention. The doses used ranged from a minimum of 280 mg/day to 1200 mg/day with a mean of approximately 600 mg/day. The safe upper limit for Vitamin E (RRR alpha-tocopherol) for adults is 300 mg/d which equates to 273 mg/d RRRDL-alpha-Tocopherol Acetate.

A dose of 250 mg/d RRRDL-alpha-Tocopherol Acetate ensures that the UL (upper limit) is not exceeded in those with an intake of Vitamin E from foods recommended daily, for example an intake which is 10 mg/d in men and 8 mg/d in women.

Selenium improves the activity of antioxidant selenoenzymes and minimizes DNA damage caused by reactive oxygen species.

Selenium (Se) is an essential micronutrient for humans, acting as a component of the unusual amino acids, selenocysteine (Se-Cys) and selenomethionine (Se-Met). Despite its very low level in humans, Selenium plays an important and unique role among the (semi)metal trace essential elements because it is the only one for which incorporation into proteins is genetically encoded, as the constitutive part of the 21st amino acid, selenocysteine. Twenty-five selenoproteins have been identified so far in the human proteome. When Se levels are low, the cell cannot synthesis selenoproteins, although some selenoproteins and some tissues are prioritized over others. Characterized functions of known selenoproteins, include Selenium transport (selenoprotein P), antioxidant/redox properties (glutathione peroxidases (GPxs), thioredoxin reductases and selenoprotein P) and anti-inflammatory properties (selenoprotein S and GPx4).

Selenium replaces sulfur in amino acids and proteins that alters their redox chemistry due to the inability of Selenium to form π bonds of all types. The outer valence electrons of Selenium are also more loosely held than those of sulfur. As a result, Selenium is a better nucleophile and will react with reactive oxygen species faster than sulfur, but the resulting lack of π-bond character in the Se—O bond means that the Se-oxide can be much more readily reduced in comparison to S-oxides. The combination of these properties means that replacement of sulfur with Selenium in nature results in a Selenium-containing biomolecule that resists permanent oxidation.

Interestingly an additional functionally characterized mammalian selenoprotein is methionine-R-sulfoxide reductase 1 (MsrB1). This is a zinc-containing selenoprotein that was found to function as a stereospecific methionine-Ra sulfoxide reductase, which catalyzes repair of the R enantiomer of oxidized methionine residues in proteins. This is another important role of Selenium in prevention of the aging process caused by oxidative stress.

Selenium is believed to be is essential for human well-being largely due to its potent antioxidant, anti-inflammatory, and antiviral properties. Selenium deficiency can Keshan disease that leads to muscle deterioration and cardiomyopathy.

Supplementation of tissue culture media, animal or human diets with moderate levels of certain Se compounds may protect against the formation of DNA adducts, DNA or chromosome breakage, and chromosome gain or loss.

Its organic form, selenomethionine, due to its pharmacokinetics, is likely to be more advantageous in long-term prevention, and supplementation efforts, while the inorganic form (sodium selenite) is still effective in an acute, e.g., sepsis, clinical setting.

The term “Selenium” is intended to include all functionally equivalent forms.

In the examples herein, Selenium is in the form of selenomethionine.

Selenomethionine is a naturally abundant organic form of Selenium that can also be readily converted to selenocysteine within cells. The great majority (80%) of human placebo-controlled interventions investigating effects on DNA damage prevention by Selenium used the organic seleno-methionine form of Selenium provided either as selenized yeast, selenized wheat or selenized malt, These interventions used doses of 200-300 ug/d Selenium and none reported adverse effects.

Too much Selenium is as harmful as too little. In fact, animal models show “U”-shaped efficacy curve. Thus, current recommended daily allowances differ among countries and are generally based on the amount necessary to saturate GPx enzymes. It is believed that functional selenoprotein single-nucleotide polymorphisms (SNPs) may interfere with Selenium utilization.

Observations of subjects residing in high Se regions or taking Se supplements previously identified Se intakes of up to 724 ug/d by adults as safe, although this assessment it is considered approximate. To provide a sufficiently wide margin of safety, the reference dose (RfD) or NOAEL for Se (in the USA) from all nutritional sources for a 70-kg human has been set at 350 ug/d, corresponding to 5 ug Se/(kg body per day) or 5 times the recommended dietary allowance. The dose of Se in the supplement is set at 200 ug/d which should be efficacious to optimize the genome protective effects of Selenium in those who are deficient or who have elevated DNA damage caused by excessive oxidative stress. 200 ug/d supplemental Selenium is generally considered safe and adequate for an adult of average weight subsisting on the typical American diet.

Carnosine has antioxidant, hydroxyl radical scavenger and anti-glycation effects.

Carnosine, a dipeptide composed of L-histidine and β-alanine, has numerous beneficial characteristics, such as maintaining the pH balance, antiglycation effect, antioxidant hydroxyl radical scavenger activity, and as a chelator of metal ions. It also neutralizes endogenous toxic aldehydes such as acrolein and formaldehyde that crosslink proteins and DNA causing genotoxic effects and accelerated cellular aging. Consequently, carnosine has been investigated as a treatment option for various diseases, such as ischemia reperfusion injury, chronic renal failure, diabetes, atherosclerosis and wound healing. Carnosine, in few recent studies, has also shown its beneficial effects in dementia, by improving cognitive function.

By controlling oxidative stress, suppressing glycation, and chelating metal ions, carnosine may reduce harmful sequelae such as DNA damage that is one of the hallmarks of accelerated aging. Carnosine as well as its constituent amino acid L-histidine are also powerful inhibitors of lipid oxidation as is histamine which is derive from L-histidine. There is emerging evidence that carnosine may also exert its anti-aging effects via inhibition of the mechanistic target of rapamycin (mTOR) and carbonyl scavenging.

Carnosine was first discovered in skeletal muscle, where its concentration is higher than in any other tissue. This, along with an understanding of its role as an intracellular pH buffer has made it a dipeptide of interest for the sports community particularly because recent studies suggest that its increase in muscle following beta-alanine supplementation results in improved high-intensity exercise performance and recovery.

Carnosinase 1 (CN1), a secreted dipeptidase present in blood, hydrolyzes L-Carnosine to its constituent amino acids L-histidine and β-alanine. Recently, an allelic variant of human CN1 (hCN1) was discovered that results in increased enzyme activity and is associated with susceptibility for diabetic nephropathy in human diabetic patients.

Conversely, a CNDP1 polymorphism associated with low CN1 activity correlates with significantly reduced risk for diabetic nephropathy, especially in women with type 2 diabetes, and may slow progression of chronic kidney disease in children with glomerulonephritis. Carnosine can be synthesized in muscle and brain from L-histidine and β-alanine and explains why supplementation with β-alanine alone is sufficient to raise carnosine level in muscle.

At the cellular level, carnosine prevents telomere shortening and senescence in vitro in human fibroblasts. Antioxidant effects of carnosine have benefits across several organs. In particular, the presence of high concentration of carnosine in muscle and brain suggests its importance in tissues with a high demand for aerobic respiration and that carnosine deficiency may be a cause of accelerated aging in these tissues by weakening their antioxidant defenses. The anti-glycating and lipid oxidation preventive effects of carnosine could also be of benefit to prevention of diabetes and cardiovascular diseases.

The term “carnosine” is intended to include all functionally equivalent forms

In the examples herein, carnosine is in the form of L-Carnosine, a natural form of carnosine found in foods such as beef or chicken. L-Carnosine is bio-efficacious across various health parameters mostly relating to aging and metabolic disease.

Curcumin (1,7-bis(4-hydroxy-3-methoxyphenyl)-1,6-heptadiene-3,5-dione) has several pharmacologic effects, including anti-inflammatory, antioxidant and anti-cancer activities. The molecular mechanisms underlying the targets of curcumin are diverse and involve combinations of multiple signaling pathways. Curcumin regulates several molecules in cell signal transduction pathways. Modulation of cell signaling pathways through the pleiotropic effects of curcumin likely activate cell death signals and induce apoptosis in cancer cells, thereby inhibiting the progression of disease.

The underlying mechanisms of the health effects of curcumin are diverse and appear to involve the regulation of various molecular targets, including transcription factors (such as nuclear factor-kB), growth factors (such as vascular endothelial cell growth factor), inflammatory cytokines (such as tumor necrosis factor, interleukin 1 and interleukin 6), protein kinases (such as mammalian target of rapamycin, mitogen-activated protein kinases, and Akt) and other enzymes (such as cyclooxygenase 2 and 5 lipoxygenase). The capacity of curcumin to attenuate inflammation is reflected, for example, by its ability to down-regulate at least eleven transcription factors (such as Ets, NFAT, NFκβ) that promote transcription of tumor necrosis factor.

Furthermore, curcumin is known to be an antioxidant, as it can scavenge free radicals from biological media. For example, the hydrogen atom of the OH group attached to the phenol moiety of curcumin is most efficiently abstracted by an OH radical.

Curcumin has anti-inflammatory and health promoting effects, including improving oxidative and inflammatory status in patients with Metabolic Syndrome. Curcuminoids natural, safe and effective CRP-lowering agents. Curcumin also lowers circulating TNF-α concentration is used in the treatment of arthritis.

It has been found that a dose of 1 g per day is sufficient to improve plasma total antioxidant capacity and reduce fasting glucose and triglycerides in diabetics.

Antioxidant, cancer growth inhibition, and cellular rejuvenation effects can occur at concentrations of 20-50 millimolar carnosine.

Curcumin has a protective role against free-radical-mediated peroxidation of membrane lipids and oxidative damage of DNA. It also has anti-inflammatory effect by inhibition of COX-2, LOX, and iNOS.

The term “curcumin” is intended to include all functionally equivalent forms.

Curcuminoids reduce circulating C-Reactive Protein levels, lower circulating IL-6 concentrations, reduced serum LDL-cholesterol and Triglycerides levels, decrease fasting blood glucose, and decrease the concentrations of HbA1c.

In the examples herein, curcumin is in the form of curcumin with piperine. Also, in the examples, the dosage of curcumin in the formulation or composition or method is about 500 mg/day.

Hesperidin protects against oxidative stress-induced DNA damage and cyclobutane dimers caused by UV.

Hesperidin and its aglycone hesperitin have antioxidant and anti-inflammatory properties. The antioxidant activity is most likely due to free-radical scavenging as hesperidin was shown to prevent DNA breakage induced by hydroxyl radicals generated by photolysis of water or the Fenton reaction. The antioxidant activity of hesperidin is not only limited to its radical scavenging activity, but it augmented the antioxidant cellular defenses via the ERK/Nrf2 signaling pathway as well.

Apart from antioxidant and inflammatory effects hesperidin also exerts protective against risk factors for cardiometabolic diseases via other mechanisms. For example, studies in mice showed that hesperidin ameliorated high fat diet (HFD)-induced weight gain, improved insulin resistance and ameliorated hyperlipidemia and, furthermore, suppressed HFD-induced hepatic steatosis, atherosclerotic plaque area and macrophage foam cell formation. These hesperidin effects were explained mechanistically by its down-regulation of the expressions of acetyl coenzyme A carboxylase alpha (ACCα) and fatty acid synthase (FAS) which are two key enzymes in fatty acid and triglyceride synthesis in liver; and upregulation of expression of hepatic ATP-binding cassette transporters G8 (ABCG8), macrophage ATP-binding cassette transporters A1 (ABCA1) and G1 (ABCG1) which are transporters involved in the process of reverse cholesterol transport. Hesperidin also reduces oxidative stress by normalizing activities of antioxidant enzymes and inflammation in HFD-fed mice.

In humans, hesperidin intake can modulate the expression of about 1,819 genes. Many of these genes are implicated in chemotaxis, adhesion, infiltration and lipid transport, which were suggestive of lower recruitment and infiltration of circulating cells to vascular wall and lower lipid accumulation.

The term “hesperidin” is intended to include all functionally equivalent forms.

In the examples, hesperidin is in the form of hesperidin (pure).

Lycopene in human in vivo and in vitro studies, reduced inflammatory signals and prevented oxidative DNA damage and lipid peroxidation.

Lycopene is a lipid soluble carotenoid present in abundance in tomatoes and tomato paste products. It is primarily known for its antioxidant and anti-inflammatory properties. It exerts these beneficial effects and others by upregulating expression of Nrf2, a master regulator of detoxification and also antioxidant, anti-inflammatory and other cytoprotective mechanisms. For example, lycopene inhibits NF-κB activation and adhesion molecule expression through Nrf2-mediated heme oxygenase-1 in endothelial cells.

The term “lycopene” is intended to include all functionally equivalent forms.

In the examples herein, lycopene is in the form of lycopene (pure).

Lutein has blue light filtering and antioxidant properties. Lutein also decreases the level of intracellular H₂O₂ by scavenging H₂O₂ thereby reducing systemic inflammation.

The term “lutein” is intended to include all functionally equivalent forms. In the examples herein, lutein is in the form of trans-lutein (pure).

Nutritional supplement compositions according to the present disclosure can be manufactured into a variety of forms, including solid and liquid dosage forms. Nonlimiting examples of such forms include tablets, caplets, liquid-filled soft capsules, suppositories, solutions and syrups.

In present examples, the nutritional supplement compositions are suitable for oral use, including, as tablets, lozenges, hard or soft capsules, aqueous or oily suspensions, emulsions, dispersible powders or granules, syrups or elixirs.

Oral compositions can have one or more coloring, sweetening, flavoring and/or preservative agents.

For a tablet formulation, suitable pharmaceutically acceptable excipients can include inert diluents such as lactose, sodium carbonate, calcium phosphate or calcium carbonate. A tablet formulation can also include: granulating and disintegrating agents such as corn starch or algenic acid; binding agents such as starch; lubricating agents such as magnesium stearate, stearic acid or talc; preservative agents such as ethyl or propyl p-hydroxybenzoate; and antioxidants such as ascorbic acid.

Tablet formulations can be uncoated or coated. A coating can modify disintegration of the tablet and the subsequent absorption of the active ingredient in the gastrointestinal tract, or to improve their stability and/or appearance. The tablet formulations can use conventional coating agents and procedures.

Compositions in the form of hard gelatin capsules can have the active ingredient mixed with an inert solid diluent such as calcium carbonate, calcium phosphate, rice fiber, rice extract or kaolin.

Compositions in the form of soft gelatin capsules can have the active ingredient mixed with water or an oil, such as peanut oil, liquid paraffin or olive oil.

Aqueous suspensions can include, in some embodiments, the active ingredient in finely powdered form together with one or more suspending agents such as sodium carboxymethylcellulose, methylcellulose, hydroxypropylmethylcellulose, sodium alginate, polyvinyl-pyrrolidone, gum tragacanth and gum acacia.

Aqueous suspensions can include, in other embodiments, the active ingredient in finely powdered form together with one or more dispersing or wetting agents. Dispersing or wetting agents can include lecithin, condensation products of an alkylene oxide with a fatty acids like polyoxethylene stearate, condensation product of ethylene oxide with long chain aliphatic alcohol like heptadecaethyleneoxycetanol, condensation product of ethylene oxide with partial esters derived from fatty acid and a hexitol such as polyoxyethylene sorbitol monooleate, and condensation product of ethylene oxide with partial ester derived from fatty acid and hexitol anhydride such as polyethylene sorbitan monooleate.

Aqueous suspensions can also include one or more: preservatives such as ethyl or propyl p-hydroxybenzoate; antioxidants such as ascorbic acid, coloring agents, flavoring agents; and/or sweetening agents such as sucrose, saccharine or aspartame.

The composition of the present disclosure can also be a solid edible product, a powdered edible product, a liquid edible product, a flowable edible product, a spoonable edible, a pourable edible product and/or a spreadable edible product.

In some embodiments, a powder can be mixed with a liquid, such as water or milk, to produce a liquid or slurry product. In other embodiments, a powder can be directly added to a food product.

The composition of the present disclosure can also be a beverage product. Beverage products can include dairy based products and soy based products.

The edible compositions can also be dairy based products, soy based products including drinks, beverages including a ready-to-drink liquid, breads and cereal based products like pasta or noodle based products and cereal bars, cakes, biscuits, ice creams, desserts, soups, porridge-type products, a liquid produced from a soluble powdered product, bar products, confectionery, snack foods, ready-to-eat meal products, pre-packed meal products, and dried meal products, soluble powdered products, and the like.

In other examples, the nutritional supplement compositions are suitable for topical use, including as creams, ointments, gels, or aqueous or oily solutions or suspensions.

In still other examples, the nutritional supplement compositions are suitable for administration by inhalation or insufflation, including as a finely divided powder or a liquid aerosol.

In yet other examples, the nutritional supplement compositions are suitable for parenteral administration, including as a sterile aqueous or oily solution for intravenous, subcutaneous, or intramuscular dosing or as a suppository for rectal dosing.

### EXPERIMENTAL

Customized culture media was prepared. The basal level for each selected nutrient was adjusted to reflect either (a) a background of deficiency in these nutrients that was predicted to increased DNA damage, or (b) a background Replete for these nutrients that was adequate for improved maintenance of DNA integrity.

These Deplete and Replete media were designed to mimic the impact of administering a composition to a nutrient-Deplete or Replete individual, respectively, on their genome integrity.

The compositions administered were F2 (including Vitamin C, Vitamin E, Selenium, carnosine, curcumin, hesperidin, lycopene, and lutein), and F1 (including folate, Vitamin B12, Vitamin D, zinc, sulforaphane, and grape seed proanthocyanidins).

The following study design was applied for in vitro studies:

| Basal | | | | |
|---|---|---|---|---|
| medium | Level and type of Supplementation | | | |
| DEPLETE | 0% | 30% F1 | 30% F2 | 30% F1 + F2 |
| DEPLETE | 0% | 100% F1 | 100% F2 | 100% F1 + F2 |
| REPLETE | 0% | 30% F1 | 30% F2 | 3 0% F1 + F2 |
| REPLETE | 0% | 100% F1 | 100% F2 | 100% F1 + F2 |

Media Condition Numbering

| | |
|---|---|
| 1 | D 0% |
| 2 | D 30% F1 |
| 3 | D 30% F2 |
| 4 | D 30% F1 + F2 |
| 5 | D 0% (same as #1) |
| 6 | D 100% F1 |
| 7 | D 100% F2 |
| 8 | D 100% F1 + F2 |
| 9 | R 0% |
| 10 | R 30% F1 |
| 11 | R 30% F2 |
| 12 | R 30% F1 + F2 |
| 13 | R 0% (same as #9) |
| 14 | R 100% F1 |
| 15 | R 100% F2 |
| 16 | R 100% F1 + F2 |

D = Deplete nutrient background; R = Replete nutrient background
F1 = formula1, F2 = formula2, F1 + F2 = formula1 + formula2. 0% = no supplement, 30% = 30% of optimal supplement dose, 100% = 100% of optimal supplement dose

The in vitro model involved 11-day cultures of human peripheral blood lymphocytes grown under conditions equivalent to clinical deficiency and clinical sufficiency in the micronutrients provided F1 and/or F1.

White blood cells (lymphocytes) isolated from whole blood of six healthy adult donors were maintained in Deplete or Replete culture medium with different conditions of F1 and/or F2 supplementation (at 0%, 30% or 100% of optimal dose). The genome integrity status of cells exposed to the different combinations of nutrients was then determined by comparing the frequencies of DNA damage biomarkers (micronuclei, nucleoplasmic bridges, nuclear buds) and telomere length in each culture condition, together with indicators of nuclear/cellular division, necrosis and apoptosis.

Eleven outcome measures were reported and analyzed, including four biomarkers of DNA damage: Binucleates (BN) with micronuclei (MN), Total MN in BN, BN with Nucleoplasmic bridges (NPB), and BN with nuclear buds (NBud); four biomarkers of cell division: Mononucleates (Monos), Binucleates (BN), Multinucleates (Multis), and Nuclear division index (NDI, a value calculated based on frequency of Monos, BN and Multis); two markers of cell death: Apoptosis, and necrosis; and Telomere length.

Without supplementation, DNA damage can examples are shown in FIG. 1.

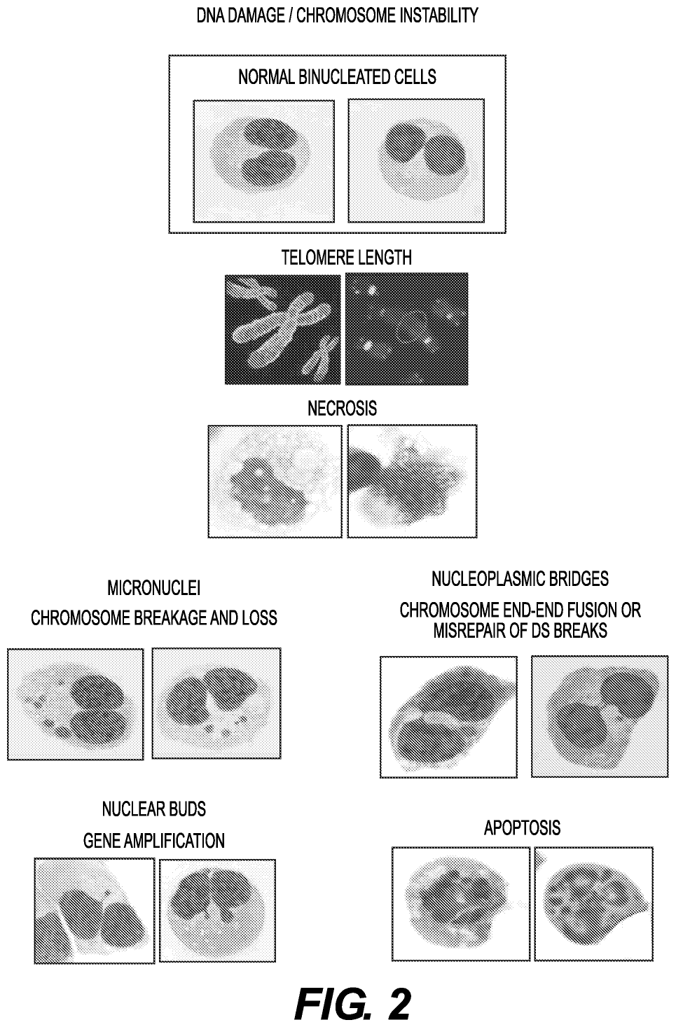

FIG. 2 summarizes the effect of supplementation with the F1, F2 and a combination of F1+F2 on BN with MN, BN with NPB and BN with NBuds at 0%, 30% or 100% of optimal dosage, in either Deplete (D) or Replete (R) basal medium. Statistical analyses were performed according to a Linear Mixed Effects Model.

Micronuclei (MN), Nucleoplasmic Bridges (NPB) and nuclear buds (NBud) are robust, validated measures of DNA damage. Higher frequencies of each has been associated (individually and in combination) with nutritional deficiency and increased morbidities including cardiovascular disease, neurodegenerative conditions, and numerous cancers.

MN are indicative of chromosome breakage or loss. During mitosis, broken DNA fragments or whole chromosomes that fail to segregate properly are packaged by the cell into discrete nuclear membranes, separate to the main nuclei. These can then be visualized and quantified as DNA damage events.

NPB originate from aberrant dicentric chromosomes formed by the misrepair of two uncapped or broken chromosomes, which can occur following loss of telomeric ends. Although NPB will break during mitosis, the CBMN-cyt assay cells are blocked at the binucleated stage, allowing visualization (and quantification) of NPB as damage events.

NBud are formed when NPB are broken unevenly during mitosis, resulting in abnormal amplification of genetic material which the cell then expels from the nucleus into the cytoplasm via the process of nuclear budding. NBud are distinguished from MN because of their residual attachment to the nucleus.

“BN with MN” indicates the percentage of binucleated cells that contain one or more micronucleus, while “Total MN” represents the actual number of MN scored, in total, in binucleated cells, in each respective data grouping.

Both measures showed greater frequency of MN in cells cultured in the un-supplemented Deplete media base compared to the Replete medium. Supplementation had no effect on MN frequency at either 30% or 100% dosage in the Replete base (p>0.05). Supplementation reduced MN frequency to optimal levels when with F1 (β=1.02, p<0.0001) or with F1+F2 (β=0.657, p=0.0043) in the Deplete medium, to a level similar to what can be seen in cells grown in Replete medium. Supplementation with F2 alone (β=−0.773, p=0.0008) significantly increased the DNA damage biomarker, over and above the “base” level seen in the Deplete media. Comparison of the median biomarker levels are shown in FIG. 2.

“BN with NPB” indicates the percentage of binucleated cells that contain one or more NPB, in each respective data grouping.

Higher frequencies of NPB were recorded in the un-supplemented Deplete medium.

Supplementation had no effect at either 30% or 100% dosage in the Replete medium (p>0.05).

However, in the Deplete medium, supplementation with F1 (β=0.741, p=0.0059) or with F1+F2 (β=0.741, p=0.0059) reduced NPB frequency to optimal levels. Supplementation with F2 did not reduce NPB in cells grown in Deplete medium.

“BN with NBud” indicates the percentage of binucleated cells that contain one or more NBud, in each respective data grouping.

Considerably higher frequencies of NBud were recorded in the un-supplemented Deplete media base. Supplementation had no effect at either 30% or 100% dosage in the Replete base medium (p>0.05). However, in the Deplete base supplementation with F1 (β=1.26, p<0.0001) or with F1+F2 (β=0.844, p<0.0001) reduced NBud frequency to optimal levels. Supplementation with only F2 did not reduce NBud in the Deplete base medium.

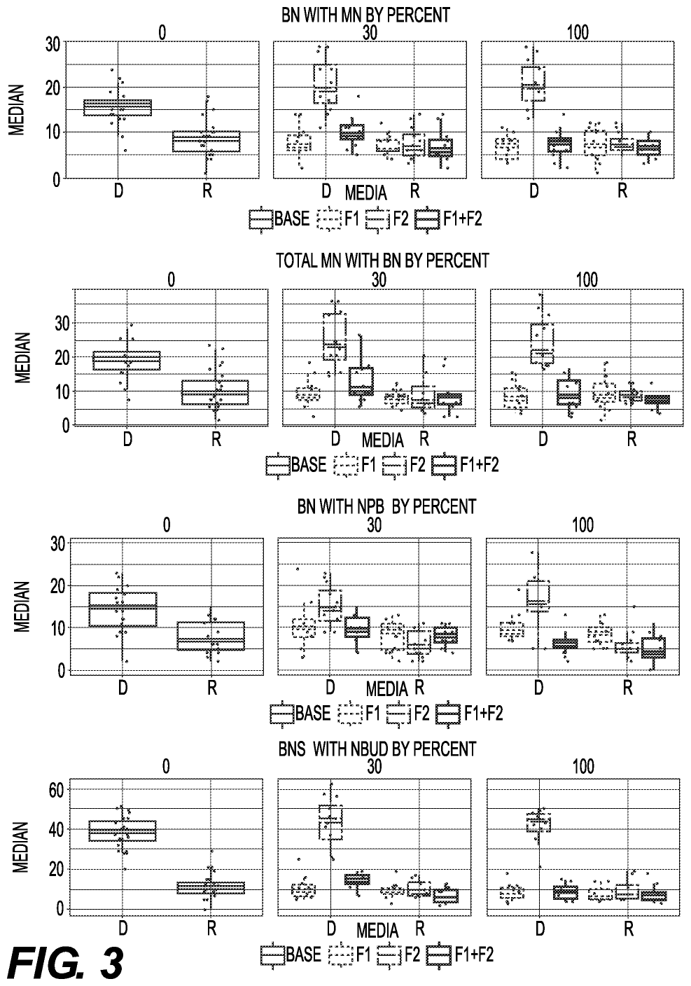

FIG. 3 shows the effect of supplementation with F1, F2 and a combination of F1+F2 at 0%, 30% or 100% of optimal dosage, in either Deplete or Replete basal medium.

Telomere length (TL) was measured by flow cytometry in viable cells at G0/G1 of the cell cycle. The result is the mean telomere content of cells from each condition, expressed as a value relative to a control cell line.

No difference in TL was observed between the un-supplemented Deplete and Replete conditions. Supplementation had no effect at either 30% or 100% dosage in the Deplete or Replete base media.

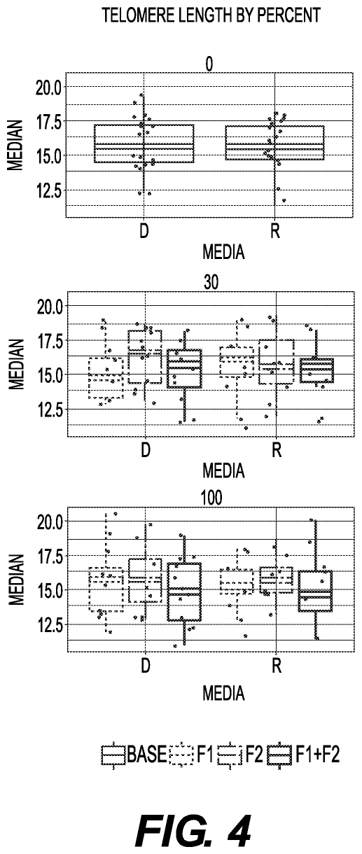

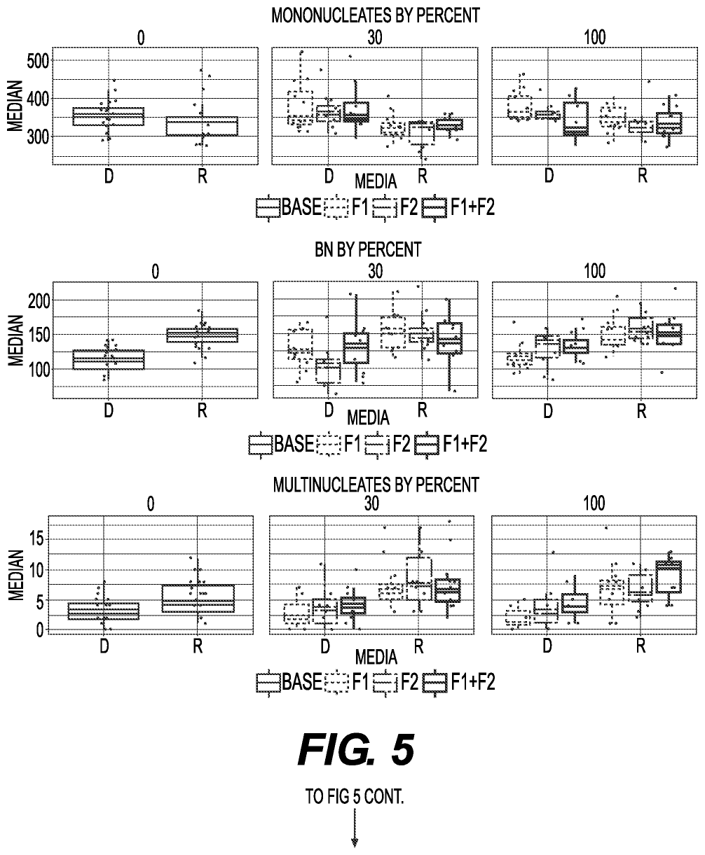

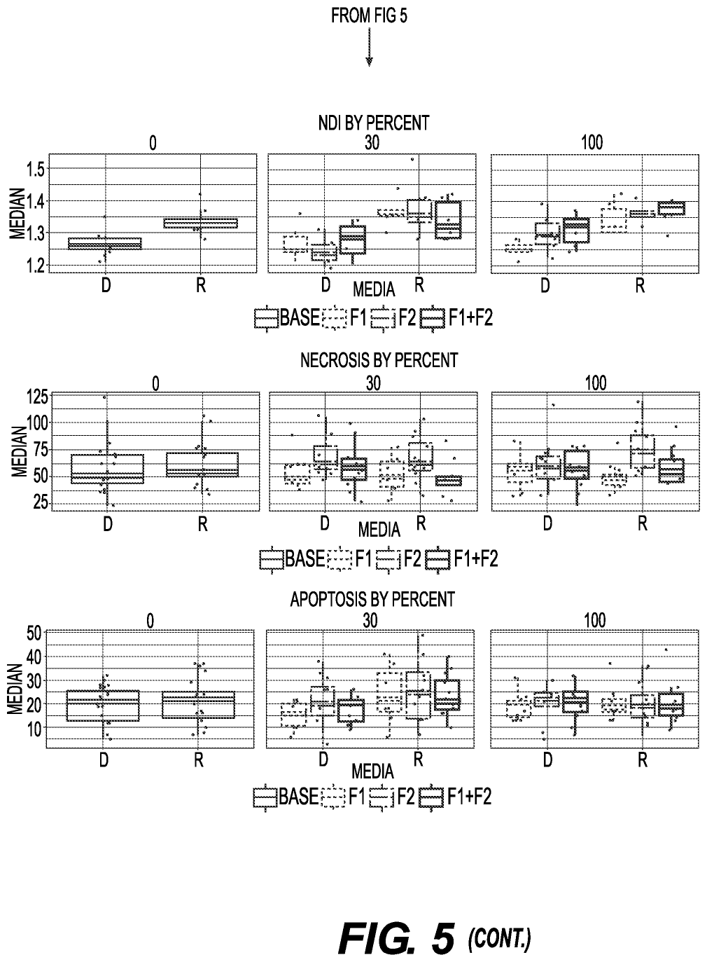

With respect to cytostasis, necrosis, apoptosis and NDI, FIG. 4 shows the effect of supplementation with F1, F2 and a combination of F1+F2 at 0%, 30% or 100% of optimal dosage, in either Deplete (D) or Replete (R) basal medium.

Cytostasis reflects the rate of cell division in each culture condition. This is quantified using the NDI, a value calculated from the relative frequencies of mononucleated cells (those that have not divided during the defined culture period), binucleated cells (once divided cells containing two nuclei), and Multinucleated cells (cells with 3 or more nuclei, indicating 2 or more rounds of cell division have occurred).

The NDI (Nuclear Division Index) is calculated using the following equation:

NDI=(M1+2M2+4M4)/N

where M1=number of mononucleated cells, M2=number of binucleated cells and M4=number of cells with more than 2 nuclei, and N is the total number of viable cells scored (excluding necrotic and apoptotic cells).

Mononucleated, Binucleated and Multinucleated Cells.

The left panels of the “Box and whisker” plots of FIG. 4 show the comparison between the 8 cultures in Deplete basal media, and the 8 cultures in Replete basal media. The observations for Monos, BN and Multis are consistent with expectation that cell division would be slower in the nutrient deficient cultures, and more prevalent in the nutrient Replete conditions.

For Monos, a main effect of F1 treatment was observed in the Deplete base condition (β=0.0697, p=0.0264). The interaction between media base and treatment was not significant, and as such little differences were expected when stratifying into either Deplete or Replete subgroups. For BN, a significant interaction was found between media base and F1+F2 treatment (p=0.027). Thus, it was expected that a different relationship would be assessed in either the Deplete or Replete media bases.

It was seen that the combination of F1+F2 treatments was significantly different as compared with no formulation on frequency of Multinucleated cells (β=0.436, p=0.0045).

Necrosis is a form of cell death caused by damage to cellular membranes, organelles and/or critical metabolic pathways required for cell survival. Apoptosis, on the other hand, is when a cell undergoes programmed cell death. The latter is less likely to lead to an immune or inflammatory response.

Basal media (D or R) and supplementation had minimal impact on necrosis. A slight increase was observed with F2 supplementation as compared with the base in the complete dataset (Main effect β=0.149, p=0.016). Stratification for media base showed significance for the F2 supplementation over the base in the Replete media only (β=0.290, p=0.00038).

Basal media (D or R) and supplementation had minimal impact on apoptosis. A slight increase was observed with F1, F2 and F1+F2 supplementation in the 30% dosage, however this was not significant.

A summary of the bioefficacy, plausibility and criticality is provided in the table below.

Summary Table of Bioefficacy, Plausibility and Criticality of Micronutrients in F2

| Micronutrient | Bioefficacy | Plausibility | Criticality |
|---|---|---|---|
| Vitamin C | medium | high | High |
| Vitamin E | Low | medium | Low |
| Selenium | medium | medium | Medium |
| Carnosine | Low | medium | Low |
| Curcumin | High | high | High |
| Hesperidin | medium | high | High |
| Lycopene | High | high | High |
| Lutein | medium | medium | Medium |

The studies conducted and the data suggest that a composition according to the present disclosure can improve DNA integrity in those who: (i) are deficient in one or more of the ingredients in the supplement due to malnutrition; (ii) are deficient in one or more of the ingredients in the supplement due to genetic defects in the absorption and metabolism of these micronutrients; (iii) are exposed to environmental genotoxins whose DNA-damaging effects can be attenuated by the micronutrients in the supplements; and/or (iv) have genetic defects in DNA replication and repair that can be attenuated by the micronutrients in the supplement.

It will be apparent to those skilled in the art that various modifications and variations can be made in the present disclosure without departing from the spirit or scope of the present disclosure. Thus, it is intended that the present disclosure covers the modifications and variations of this disclosure provided they come within the spirit or scope of the present disclosure and its equivalents.

What is claimed is:

1. A composition for oral delivery to a subject comprising Vitamin C, Vitamin E and Selenium.

2. The composition according to claim 1, further comprising one or more of: carnosine, curcumin, hesperidin, lycopene and lutein.

3. A method for lessening the rate of cellular damage to DNA of the subject comprising administering the composition of claim 2.

4. A method for reducing reactive nitrogen species (RNS) and/or reactive oxygen species (ROS) present in the subject comprising administering the composition of claim 2.

5. A method for ameliorating a cellular biomarker of DNA damage in the subject comprising administering the composition of claim 2.

6. The method of claim 5, wherein the cellular biomarker is at least one selected from the group consisting of: Micronucleus (MN) frequency in lymphocytes and buccal cells, TL in lymphocytes and buccal cells, nucleoplasmic bridges, and nuclear buds.

7. A method for increasing TL in lymphocytes and buccal cells comprising administering the composition of claim 2.

8. A method for reducing Micronucleus (MN) frequency in lymphocytes and buccal cells comprising administering the composition of claim 2.

9. A method to reduce abnormal cellular methylation of DNA comprising administering the composition of claim 2.

10. A method for increasing lipid homeostasis comprising administering the composition of claim 2.

11. A method for decreasing abnormal cell division comprising administering the composition of claim 2.

12. A method for decreasing protein oxidation comprising administering the composition of claim 2.

13. A method for decreasing DNA oxidation and breakage comprising administering the composition of claim 2.

14. A method for decreasing lipid peroxidation comprising administering the composition of claim 2.

15. A method for decreasing cellular inflammation comprising administering the composition of claim 2.
